(19) United States
(12) Patent Application Publication
(10) Pub. No.: US 2015/0038378 A1
(43) Pub. Date: Feb. 5, 2015

(54) BIOCOMPATIBLE GRAPHENE SENSOR

(71) Applicants: Mark Ming-Cheng Cheng, Novi, MI (US); Zhixian Zhou, Troy, MI (US)

(72) Inventors: Mark Ming-Cheng Cheng, Novi, MI (US); Zhixian Zhou, Troy, MI (US)

(73) Assignee: Wayne State University, Detriot, MI (US)

(21) Appl. No.: 13/985,447

(22) PCT Filed: Feb. 16, 2012
(86) PCT No.: PCT/US12/25377

§ 371 Date: Nov. 4, 2013

Related U.S. Application Data
(60) Provisional application No. 61/443,474, filed on Feb. 16, 2011.

(51) Int. Cl. G01N 27/414; A61B 5/145; G01N 33/543; A61B 5/00

(52) U.S. Cl. CPC G01N 27/4145; A61B 5/6852; A61B 5/14503; G01N 33/54386
USPC 506/39; 600/373; 204/403.01

(57) ABSTRACT

A graphene biosensor is formed on an electrically insulating substrate with a single-layer graphene sheet arranged between two metallic electrodes. The graphene sheet is in electrical contact with the metallic electrodes. The graphene sheet has perforations creating edges in the graphene sheet. The perforations may be holes on a micrometer scale or in a nanometer scale. The biosensor can be configured as an ISFET. The graphene sheet may comprise affinity probes immobilized on the edges for attaching specific molecules to the graphene sheet. Several graphene sheets may be arranged in a microarray with different affinity probes on different graphene sheets. The sensor may also be arranged on the distal end of a catheter for in situ measurements in a body vessel.

### FIELD OF THE INVENTION

The present invention relates to a biological sensor. More particular, the present invention relates to a biological sensor utilizing a field-effect transistor (FET).

### BACKGROUND OF THE INVENTION

A currently common single-protein measurement procedure in clinical diagnosis is immunoassay, in which monoclonal immunoglobulin or their antigen binding domains are used to bind antigens of interest. Among these assays, the enzyme-linked immunosorbent assay (ELISA) is the most widely used assay for the measurement of a single protein in solution. The ELISA experiment is typically conducted in a well plate of 96 or 384 wells, with a limit of sensitivity of about 1 pg/mL, a dynamic range of about 10³ (corresponding to a range of about 0.1 ng/mL-about 100 ng/mL) and over several hours of experiment time. A microarray is fabricated by the immobilization of affinity probes (typically antibodies) in arrays at high spatial density on a solid substrate. Each probe is used to capture specific proteins and then the proteins are labeled using secondary antibodies carrying fluorescent, colorimetric or radioisotope signals. The concentration of antigens is quantified by measuring the fluorescent intensity, a detection method that is similar to DNA microarrays.

Different to label-based detection, the label-free detection measures the change in inherent properties caused by the molecular binding on the sensor surface, such as charge, mass, stress, or dielectric constant, etc. The label-free techniques not only eliminate laborious, time-consuming procedures for tagging fluorescent probes, but also enable the determination of reaction kinetics of biomolecular interaction in real-time. Among these developments, detection of biomolecular charges using nanowires is very attractive because it does not require sophisticated instrumentation, and offers high sensitivity and a broad detectable dynamic range. To date, most of antibody conjugation on nanowires is still conducted by pipette or microfluidic channels. The high cost of reproducibly manufacturing nanowires and the lack of reliable, high-throughput surface functionalization hinder the applications of nanowires to label-free detection for cancer diagnosis and point-of-care testing.

It is therefore desirable to find a cheaper and faster way of biomedical and chemical sensing that is suitable for clinical applications.

### SUMMARY OF THE INVENTION

According to the present invention, a graphene biosensor comprises an electrically insulating substrate, a first metallic electrode and a second metallic electrode, the first and second metallic electrodes being mounted on the substrate, a single-layer graphene sheet in electrical contact with and connecting the first and second metallic electrodes. The graphene sheet comprises perforations with edges having a total edge length. The added edge length enhances the reactivity of the graphene sheet with biomolecules.

According to one aspect of the invention, the perforations are formed by holes in the graphene sheet or gaps between graphene strips.

According to one aspect of the invention, the holes or gaps have a diameter smaller than about 10 μm, preferably smaller than 10 μm.

According to one aspect of the invention, the holes or gaps have a diameter smaller than about 5 μm, preferably smaller than 5 μm.

According to one aspect of the invention, the perforations are arranged in a substantially regular pattern at a distance from each other smaller than about 5 μm, preferably smaller than 5 μm.

According to one aspect of the invention, the perforations are arranged in a substantially regular pattern at a distance from each other smaller than about 1 μm, preferably smaller than 1 μm.

According to one aspect of the invention, the total edge length relative to the graphene sheet area has an edge-to-area ratio greater than about 0.1 μm⁻¹, preferably greater than 0.1 μm⁻¹.

According to one aspect of the invention, the edge-to-area ratio is greater than about 0.5 μm⁻¹, preferably greater than 0.5 μm⁻¹.

According to one aspect of the invention, the edge-to-area ratio is greater than about 0.7 μm⁻¹, preferably greater than 0.7 μm⁻¹.

According to one aspect of the invention, the biosensor further comprises a reference electrode configured to be supplied with a variable gate voltage and configured to be in indirect contact with the graphene sheet via a fluid connection.

According to one aspect of the invention, graphene sheet may comprise immobilized affinity probes configured to attach specific molecules to the graphene sheet. The affinity probes may be antibodies configured to attach specific antigens to the graphene sheet. These specific antibodies allow for selective testing for biomarkers.

According to another aspect of the invention, the sensor may include an array of at least two graphene sheets including a first and a second graphene sheet. Several graphene sheets of the array may comprise affinity probes.

According to a further aspect of the invention, different affinity probes may be associated with different graphene sheets.

According to yet another aspect of the invention, at least one of the graphene sheets in the array may comprise immobilized affinity probes, and at least one of the graphene sheets may be free of any affinity probes.

According to one aspect of the invention, the sensor may be configured as an ion-sensitive field effect transistor (ISFET) for testing fluids. The sensor may be configured to measure a property of a liquid contacting the reference electrode, the source electrode, the drain electrode and the first graphene sheet, and the sensor may measure an electric current between the drain electrode and the source electrode during exposure to the liquid.

According to another aspect of the invention, the sensor may be calibrated to operate near the Dirac point of the conductance during exposure to the liquid.

In a further development of the invention, the ISFET may comprise a cavity and at least two ports in fluid communication with the cavity. The cavity may contain the graphene sheet and the set of electrodes, and the ports may be configured to supply the liquid to the cavity and to drain the liquid from the cavity.

According to a further aspect of the invention, for continuous measurement over a period of time, one of the at least two ports may be an inlet port for supplying the liquid to the cavity and the one of the at least two ports maybe an outlet port for draining the liquid from the cavity, and both inlet port and outlet port may be configured to be operated at the same time to allow a continuous flow of liquid through the cavity.

According to yet another aspect of the invention, the sensor may be configured to be mounted on a catheter of the type having a proximal end and a distal end, an electric connector disposed at the proximal end; and an electrical connection extending along the catheter and connecting the distal end to the electric connector. The sensor may be configured to be mounted on the distal end and to be connected to the electrical connection for in situ measurements. The graphene sheet of the sensor configured to be mounted on the tip of the catheter may also carry immobilized affinity probes configured to attach specific molecules to the graphene sheet.

Further details and benefits become apparent from the following description of various embodiments of the invention in connection with the attached drawings.

### BRIEF DESCRIPTION OF THE DRAWINGS

FIG. 1 is a schematic depiction of a graphene sheet;

FIG. 2 schematically shows the basic structure of an ion-sensitive field effect transistor (ISFET) comprising a graphene sheet in accordance with one embodiment of the present invention;

FIGS. 3a through 3i schematically show assembly steps of an ISFET suitable for the analysis of liquids in accordance with one embodiment of the present invention;

FIG. 4 shoes a graph of the drain-source current through a substantially homogenous graphene sheet over the gate voltage in an ISFET;

FIG. 5 shows a plot of the gate voltages of the Dirac points of FIG. 4 over the pH values of the tested fluids;

FIG. 6 shows a graph of the diode current through a graphene sheet with inserted edge defects over the gate voltage in an ISFET;

FIG. 7 shows a plot of the gate voltages of the Dirac points of FIG. 6 over the pH values of the tested fluids;

FIGS. 8a through 8d show steps of preparing a graphene sheet for detecting and identifying specific biomolecules in accordance with one embodiment of the present invention;

FIG. 9 schematically shows a structure of an ISFET for detecting the specific biomolecules in accordance with one embodiment of the present invention;

FIG. 10 shows a graph of the conductance of a graphene sheet prepared for detecting bovine serum albumin (BSA) in an ISFET over the gate voltage;

FIG. 11 schematically shows steps of preparing and evaluating a microarray of a plurality of graphene sheets for detecting a plurality of different biomolecules in accordance with one embodiment of the present invention;

FIG. 12 shows a catheter utilizing an ISFET for detecting biological or chemical conditions inside a body in accordance with one embodiment of the present invention;

FIG. 13 shows an environmental partial view of a catheter tip with an ISFET inserted into a blood vessel;

FIGS. 14a through 14h show manufacturing steps of making a graphene sheet with edge defects according to one embodiment of the invention;

FIG. 15 shows a graphene sheet with added edge defects according to one embodiment of the invention; and

FIG. 16 shows a graphene sheet with added edge defects according to a further embodiment of the invention;

### DETAILED DESCRIPTION OF THE DRAWINGS

The appended drawings serve purely illustrative purposes and are not intended to limit the scope of the present invention.

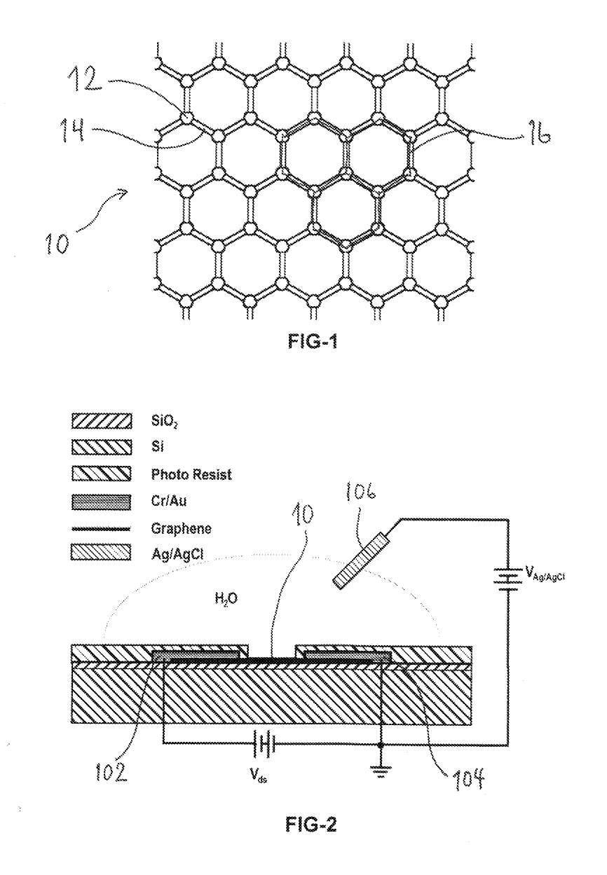

FIG. 1 shows a graphene sheet 10. As shown, graphene is a flat single atomic layer of carbon atoms 12. In graphene, the electrons of the s orbital and of two of the three p orbitals in the outer electron shell form three sp2 hybrid orbitals arranged in one common plane at 120° angles with respect to each other. These hybrid sp2 orbitals each form a bond with a hybrid sp2 orbital of another carbon atom 12 so that each carbon atom 12 forms bonds with three other carbon atoms 12 via the sp2 hybrid bonds 14. The resulting planar arrangement of carbon atoms 12 forms a hexagonal honeycomb lattice 16. Graphene has exceptional electronic, mechanical and chemical properties. Moreover, graphene is a semiconductor with zero bandgap, where adsorbed chemical and biomolecules can be translated into an electrical signal by changing the conductivity of the device.

FIG. 2 shows a sensor 100 utilizing the graphene sheet 10 disposed in a cavity 108 in accordance with one embodiment of the present invention. In this embodiment, the sensor 100 is structured as an ion-sensitive field effect transistor (ISFET) with three electrodes 102, 104, and 106. In one embodiment, the electrodes 102 and 104 are made of chromium-gold deposits, and the electrode 106 is made of silver and silver chloride. Presuming the charge carriers of the ISFET are holes, the electrode 102 operates as a source electrode and electrode 104 operates as a drain electrode. Where the charge carriers are electrons, source and drain are reversed. Electrode 106 is a reference electrode supplying a variable gate voltage. In the following, the reference electrode 106 will be interchangeably called gate electrode.

The electrodes 102 and 104 are in direct electrical contact with the graphene sheet 10. A drain-source voltage V_{ds} is applied between the source and drain electrodes 102 and 104, and a gate voltage V_{Ag/AgCl} is applied between the reference electrode 106 and the drain electrode 104. The reference electrode 106 is not in direct contact with the graphene sheet 10 and supplies the variable gate voltage to a water-based liquid, referenced as H₂O, that is in contact with all three electrodes 102, 104, and 106, as well as the graphene sheet 10. Thus, in this embodiment, the electrode 106 is only in indirect contact with the graphene sheet 10 and with the other two electrodes 102 and 104 via the liquid. None of the electrodes 102-106 are in direct electrical contact with each other. The sensor 100 measures the conductance of the graphene sheet 10 by measuring an electric current between the electrodes 102 and 104 under varying gate voltages supplied by the reference electrode 106.

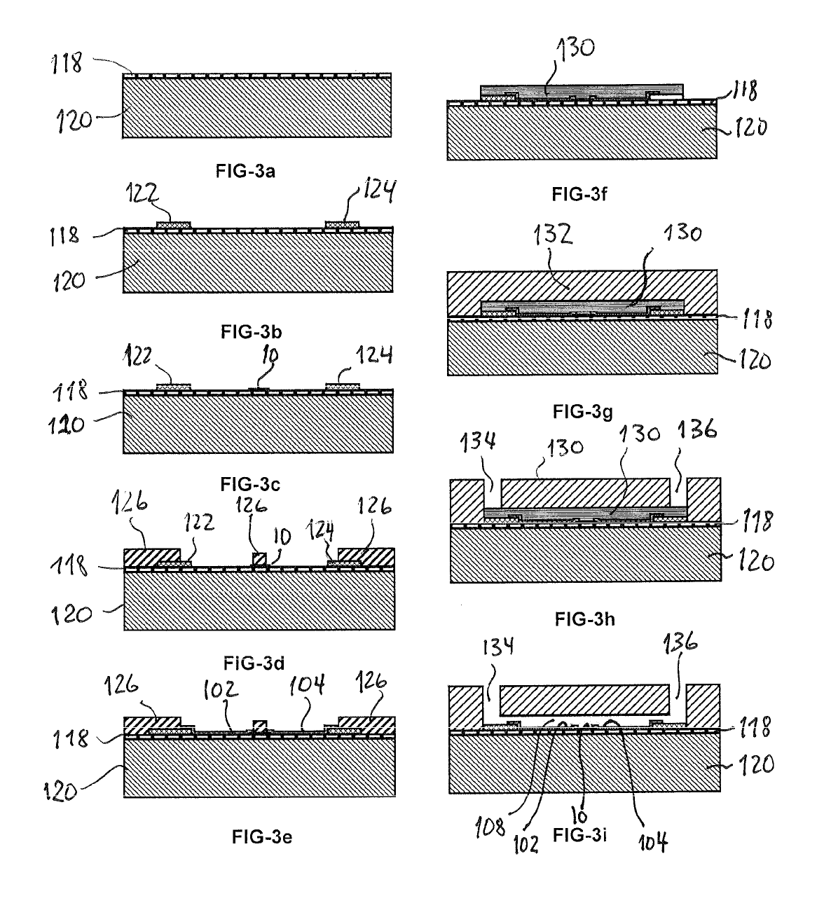

FIGS. 3a through 3i show assembly steps for the manufacture of an ISFET similar to the embodiment of FIG. 2 in accordance with examples of the present invention. A commonly used method to produce graphene is mechanical exfoliation, generally called Scotch tape method. This method produces small amounts of high-quality graphene samples that are suitable for fundamental study. The starting material may be highly oriented pyrolytic graphite (HOPG). Graphite flakes can be attached to an adhesive tape and repeatedly exfoliated. The resulting single graphene layer can then be transferred to a clean silicon substrate. This method produces small amounts of high-quality graphene samples.

For example, for larger graphene samples, a chemical vapor deposition (CVD) system can be applied to grow wafer-scale graphene. In the CVD method, a thin copper foil with a thickness of about 25 μm, preferably 25 μm, may first be thermally annealed at a high temperature ranging from about 900° C. to about 1000° C., preferably between 900° C. and 1000° C. The copper can subsequently be exposed to hydrocarbon environment in a CVD chamber exposed to a flow of methane (CH₄). Preferred values for the conditions inside the CVD chamber are about 30 standard cubic centimeters per minute (about 30 sccm), preferably 30 sccm, for the flow of CH₄, a pressure of about 500 mTorr, preferably 500 mTorr, and a temperature of about 1000° C., preferably 1000° C. Next, the graphene-covered copper substrate can be spin-coated with a polymer film. In the present example, the polymer is Poly(methyl methacrylate) (PMMA), a transparent thermoplastic with various uses, for example as a glass substitute or as photoresist for e-beam lithography. Subsequently, the copper foil can be etched as a sacrificial layer using FeCl3. Dissolving the PMMA film in Acetone results in a single graphene layer that can be transferred onto a wafer.

Once a graphene sheet 10 has been produced, a graphene sensor can be manufactured, for example through the steps illustrated in FIGS. 3a through 3i.

As illustrated in FIG. 3a, a silicon dioxide (SiO₂) layer 118 of about 300 nm thickness, preferably 300 nm, can be grown on a silicon wafer 120 by thermal oxidation. The SiO₂ layer forms an electric insulator.

Then, according to FIG. 3b, electrode contacts 122 and 124 can be deposited by e-beam evaporation by generating a chromium deposit of about 5 nm thickness, preferably 5 nm, and a gold deposit of about 50 nm thickness, preferably 50 nm, in the locations of electrode contacts 122 and 124.

As illustrated in FIG. 3c, the graphene sheet 10 can then be deposited on the SiO₂ layer 118 of the silicon wafer 120, for instance by stamping. The graphene sheet may have been obtained by the scotch tape method or by the CVD process. As new processes of manufacturing graphene sheets become available, any of such methods that produce suitable graphene monolayers may be used to produce the graphene sheet 10. After applying the graphene sheet 10 on the SiO₂ layer, the assembly may be cleaned to remove tape residue or other contaminating deposits.

Now referring to FIG. 3d, a thermoplastic layer 126, for instance poly(methyl methacrylate) (PMMA), may be applied on top of the electrode contacts 122 and 124 and the graphene sheet 10. Electrode patterns 128 can then be created with e-beam lithography by removing portions of the thermoplastic layer 126 in the shape of the electrodes 102 and 104.

Now referring to FIG. 3e, Cr/Au layers can then be deposited in the electrode patterns 128 to form the electrodes 102 and 104 using e-beam evaporation in analogy to the deposit of the electrode contacts 122 and 124. In comparison to carbon nanotubes, graphene forms a more robust and more reproducible ohmic contact with Cr—Au electrodes, which supports ballistic electron transportation. After formation of the electrodes 102 and 104, the thermoplastic layer 126 has served its purpose and can be removed with a lift-off process.

Now referring to FIG. 3f, the resulting structure may be coated with a photoresist layer 130, which, for example, is available under the commercial name SU-8. The shape and volume of the photoresist layer 130 defines the shape and volume of the chamber 108 of the sensor 100.

Now referring to FIG. 3g, a silicone block 132, for instance Polydimethylsiloxane (PDMS), can be poured onto the structure and cured. PDMS cures at room temperature over several hours. As shown in FIG. 3h, holes forming an inlet 134 and an outlet 136 can be formed with a stiff punch.

As illustrated in FIG. 3i, the photoresist layer 130 can subsequently be removed, resulting in a chamber 108. The chamber 108 constitutes a microscopical flow channel for liquid entering through the inlet 134 and exiting through the outlet 136. Notably, inlet 134 and outlet 136 can be swapped without limitation. Finally, the silicone block 132 can be bonded with the SiO₂ layer 118 of the silicon wafer 120 for sealing the chamber 108. This completes the process of manufacturing the sensor 100 operating as an ISFET.

Experiments have shown that graphene containing a large number of impurities and structural defects exhibits a bad signal-to-noise ratio. Accordingly, it was believed that graphene with a highly regular structure is ideal for biosensors. Chemical vapor deposition has enabled the creation of such regular structures.

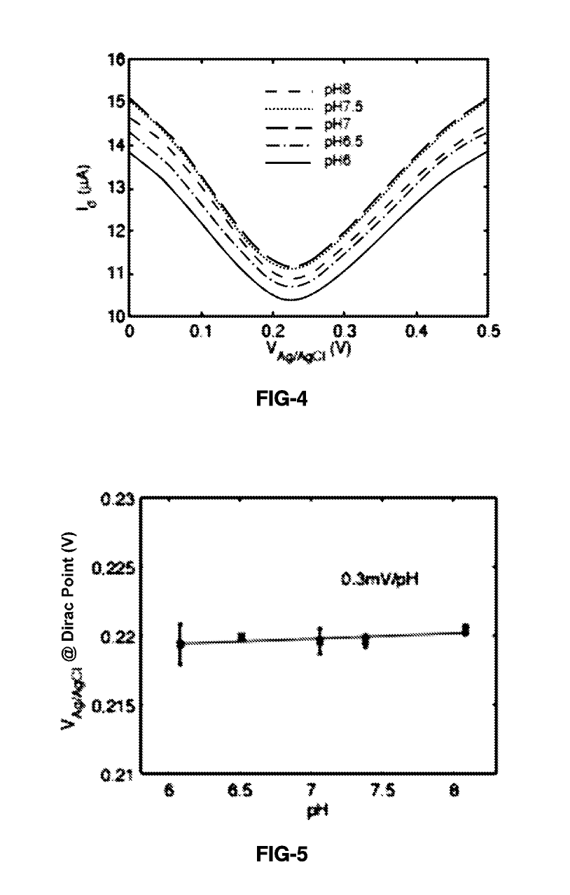

FIG. 4 is a graph showing five different current curves as a function of a gate voltage applied to an ISFET 100 with a substantially homogenous graphene sheet 10. Homogenous in this context means that the graphene sheet was generated with a very regular honeycomb structure by chemical vapor deposition. Phosphate buffer solutions with incrementally differing pH values can be injected into the microfluidic channel formed by chamber 108 at a flow rate of about 0.1 mL/min, preferably 0.1 mL/min, via a syringe pump. The pH of the fluids are changed about every five minutes, preferably every five minutes, and the conductance of the graphene sheet 10 can be monitored by measuring the drain-source current I_{d} flowing between the source electrode 102 and the drain electrode 104. In the solution-filled chamber 108, the electrical double layer at interfaces between the graphene sheet 10 and the phosphate buffer solution acts as a top gate insulator with an approximate electrostatic gate capacitance Gₑₗ of about 500 nF/cm², preferably 500 nF/cm², depending on ionic strength.

The sensor has the configuration of an ISFET (ion-sensitive field effect transistor), and the carrier concentration (conductance) can be modulated by the applied gate voltage and ionic groups in the solution. The graphene sheet 10 is a p-type semiconductor under the ambient conditions with holes constituting the charge carriers, where impurities can be partly attributed to adsorbed oxygen molecules or residues of photoresist. The point of minimum conductivity is called Dirac point, where charge carriers change from holes to electrons. When measuring drain-source current I_{d} through substantially homogenous graphene sheet 10, the Dirac points are nearly indistinguishable for all pH values. As illustrated in FIG. 5, a plot of the Dirac voltages for all measured pH values results in a nearly constant line.

This behavior can be explained with the hydrophobic properties of homogenous graphene. The regular honeycomb structure resists electric polarization and will thus not react easily with polar biomolecules that may, for example, contain OH groups. It has been discovered that artificial, controlled edge defects in the otherwise highly regular graphene sheet structure enhance the reactivity of the graphene sheet 10.

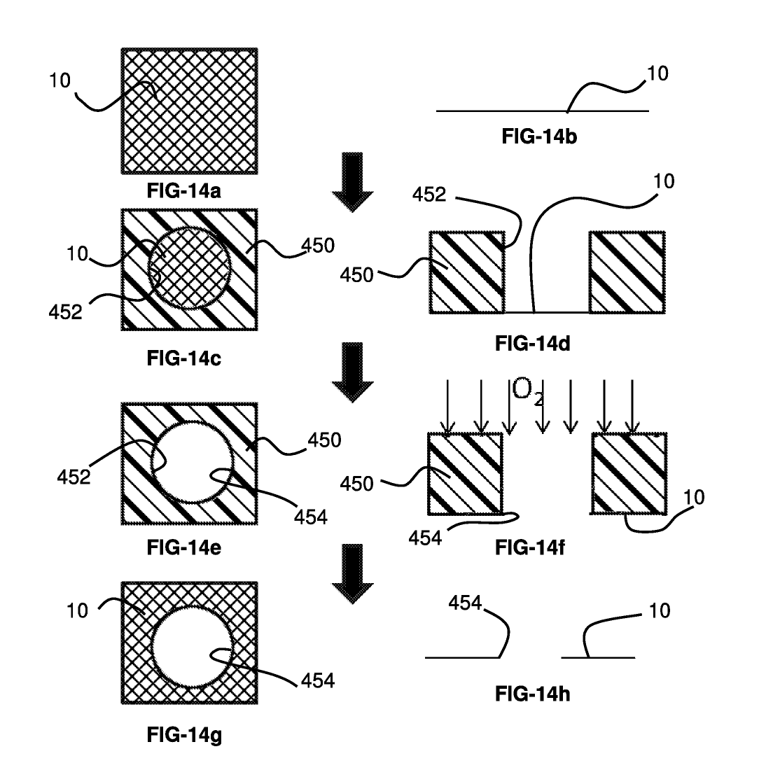

FIGS. 14a through 14h show steps in a process of creating edge defects in a highly regular graphene sheet according to one embodiment of the invention. A large graphene sheet 10 can be grown, for example by the CVD method using CH4 as reaction gas on a clean copper foil. Initially, a copper foil can be annealed for increased homogeneity and polished to obtain a surface even enough for creating a very regular honeycomb pattern of graphene. CH4 can be flown into the furnace at a rate of about 30 sccm, preferably 30 sccm, for about 30 minutes, preferably 30 minutes, at about 1000° C., preferably 1000° C., and the pressure can be controlled to be about 500 mTorr, preferably 500 mTorr. After deposition of the carbon on the copper foil, the copper foil can be removed via wet etching to dissolve the copper foil. This leaves a substantially pure graphene sheet that can be transferred onto a SiO2/Si substrate of about 300 nm thickness, preferably 300 nm, by stamping as described in connection with FIG. 3. Cr/Au electrodes can created using optical lithography on two layer lift-off resist and deposited using e-beam evaporation. After depositing Cr—Au electrode contacts, the steps of FIG. 14 can be employed.

FIG. 14a shows a small cutout of a graphene sheet 10. For simplicity, the substrate supporting the graphene sheet is not shown. FIG. 14b shows the graphene sheet 10 of FIG. 14a in a side view.

As shown in FIGS. 14c and 14d, the graphene sheet can be coated with a photoresist layer 452, for example PMMA. A very fine pattern can be defined in the photoresist layer 452 with electron beam lithography (e-beam lithography) to create voids 452 exposing the graphene sheet 10 to the environment.

As shown in FIGS. 14e and 14f, the graphene sheet 10 can subsequently be perforated by exposing the structure to oxygen plasma. In the locations of the voids 452, the graphene sheet is destroyed, leaving a fine pattern of holes 454 in the graphene sheet 10. while the holes of this embodiment are roughly circular, it is evident that edge defects can be introduced in different shapes and even in other ways, for instance by using graphene strips with gaps between them.

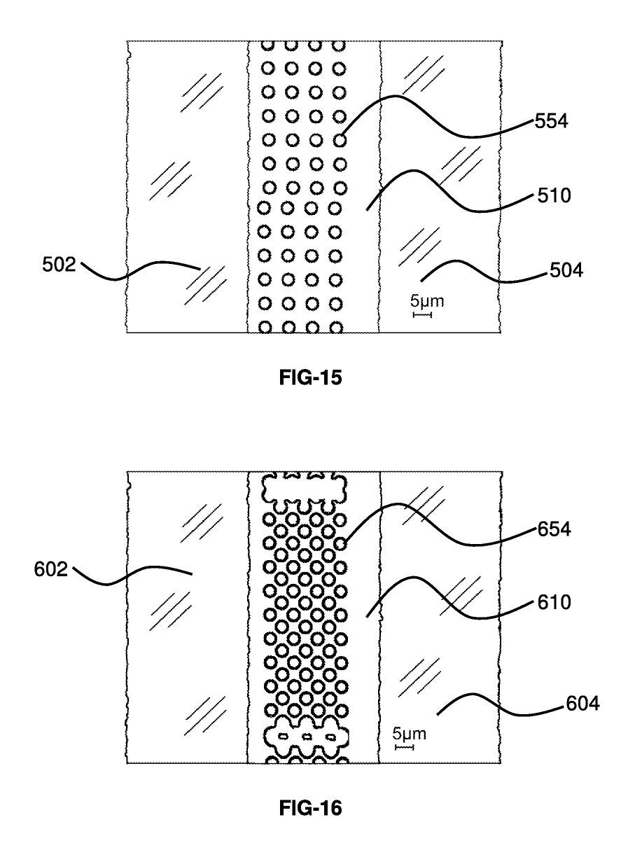

FIGS. 15 and 16 show two embodiments of the invention, in which graphene sheets 510 and 610 are perforated with fine hole patterns. On the left and right side of FIG. 15, strips of Cr—Au electrodes 502 and 504 are visible. Between the electrodes 502 and 504, the graphene sheet 510 shows holes 554 of substantially equal size in a substantially regular arrangement. The holes 554 have predominantly equal distances to each other. The arrangement of the holes 554 creates edges in the graphene sheet 510 along the peripheries of the holes 554. These edges provide artificial, controlled, edge defects in the honeycomb pattern that promote interaction between the graphene sheet and polar biomolecules. The holes 554 of the shown embodiment have a size and distance from each other in an order of magnitude of single-digit micrometers. With suitable technology, for example nanolithography, perforations can be provided on a much smaller nanometer scale.

In the embodiment of FIG. 16, strips of Cr—Au electrodes 602 and 504 are visible on the left and right side of the graphene sheet 610. Between the electrodes 602 and 604, the graphene sheet 610 shows holes 654 of substantially equal size in a substantially regular arrangement. The holes 654 shown in FIG. 16 are arranged in a denser pattern than in FIG. 15, where additional holes 654 are inserted in the spaces between the locations of holes 554 shown in FIG. 15. This arrangement reduces the distance between adjacent holes 654 to less than about 1 μm, preferably less than 1 μm. The holes 654 also have predominantly equal distances to each other. Accordingly, the arrangement of the holes 654 creates even more controlled edge defects in the graphene sheet 610 along the peripheries of the holes 654 than the holes 554 in graphene sheet 510.

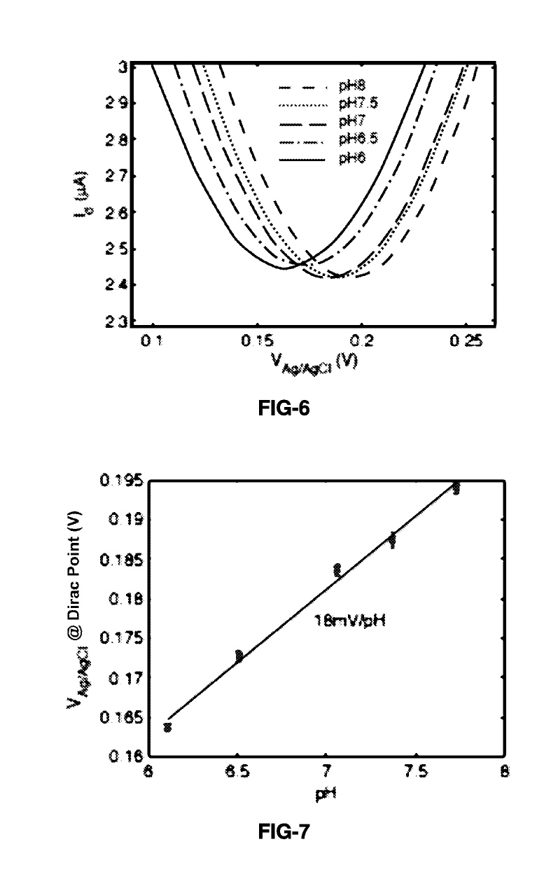

FIG. 6 illustrates calibration measurements with a graphene ISFET containing a graphene sheet patterned like the graphene sheet 610 shown in FIG. 16 under varying gate voltages for solutions with five different acidities ranging from about pH6 to about pH8, preferably pH6 to about pH8. The solutions may be physiological phosphate buffered saline solutions (PBS, about 1×, preferably 1×) with pH values adjusted using hydrochloric acid (HCl) and sodium hydroxide (NaOH). As shown in FIG. 6, the Dirac point increase is in substantially linear correlation with the pH values, which is further illustrated in FIG. 7 by plotting the Dirac point voltages over the pH values of the tested solutions. The line shown in FIG. 7 marks a good fit with the Dirac points and has a slope of about 18 mV/pH for the graphene sheet 610 of FIG. 16. The graphene sheet 610 of FIG. 16 has an edge-to-area ratio (E/A) of about 0.57 μm⁻¹, preferably 0.57 μm⁻¹. It is evident that edge defects can be introduced in other ways, for instance by using graphene strips with gaps between them. In this context, the term “graphene sheet” includes an array of graphene strips aligned next to each other with gaps between each other. The gaps are then the perforations or holes of the graphene sheet.

The edge-to-area ratio is calculated from the length of all holes 654 in an area of the graphene sheet 610 divided by the area. Other ranges of edge-to-area ratios can be obtained by changing the density, the shape, or the size of the holes 554 fabricated in the graphene sheet 610. Feasible edge-to-area ratios range from about 0.1 μm⁻¹, preferably from 0.1 μm⁻¹, to as high as is realizable within given technological and financial constraints. By using nanolithography, for example, much smaller holes can be produced at a much higher density so that edge-to-area ratios of greater than about 1 μm⁻¹, preferably greater than 1 μm⁻¹, can be produced. With the described electron beam lithography and oxygen plasma treatment, edge-to-area ratios of more than about 0.7 μm⁻¹, preferably greater than 0.7 μm⁻¹ are realistically producible.

Increased conductance is observed at the Dirac point for lower pH values, i.e. higher acidity. This increased conductance can be attributed to the increased number of negative-charged hydroxyl groups around graphene. The attached hydroxyl oxide acts as electron acceptor. Because the device is a p-type semiconductor, the charge carriers are holes, and the concentration of charge carriers increases with higher pH values. In comparison with FIG. 5, FIG. 6 shows a nearly linear correlation between pH value and Dirac point due to the artificial edge defects. While impurities and surface defects in the honeycomb pattern generate an inferior signal-to-noise ratio, controlled artificial edge defects significantly improve the operational properties of the graphene sensor due to enhanced reactivity with hydrophilic biomolecules.

In this embodiment, the minimum carrier concentration in graphene is measured in a decimal order of magnitude of 10¹² cm⁻². Such low carrier concentration makes graphene a promising sensing material. The sensitivity to biomolecules is represented by the following equation:

Sensitivity=ΔG/G=Δn/n

where G represents the conductance of graphene and n represents the number of charge carriers of graphene.

It is evident, that a small number of charge carriers in the graphene, i.e. a low conductance, results in a higher sensitivity because a given change Δn in charge carriers makes a greater difference relative to the existing number of charge carriers.

For example, detection of single gas molecules has been demonstrated using micron-sized graphene without the need for nanolithography to scale down geometry. The high electron mobility (at least about 15000 cm²V⁻¹s⁻¹, preferably at least 15000 cm²V⁻¹s⁻¹ at room temperature) of graphene leads to low Johnson noise (thermal noise) and to a rapid signal transduction for chemical and biological sensing. It is therefore possible to measure the acidity of a liquid sample according the curve shown in FIGS. 4 and 5 by determining the Dirac point of the graphene in that liquid sample.

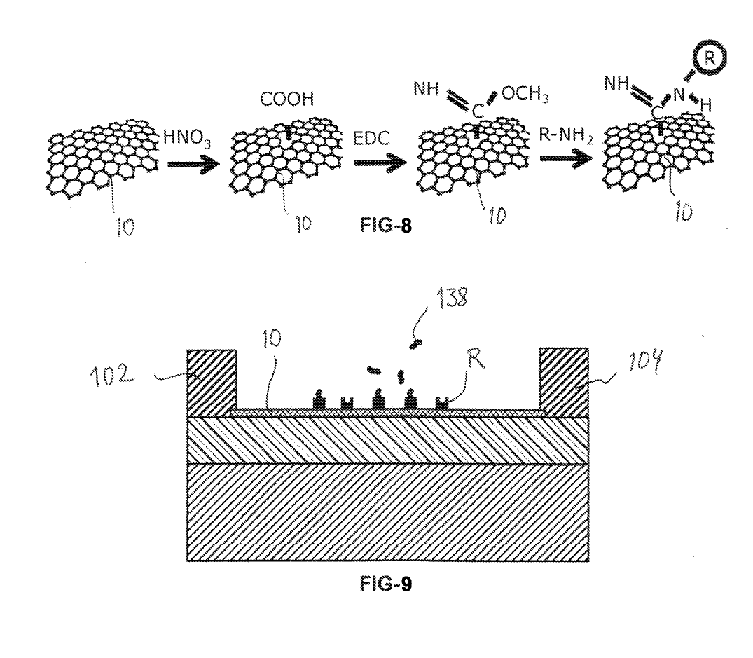

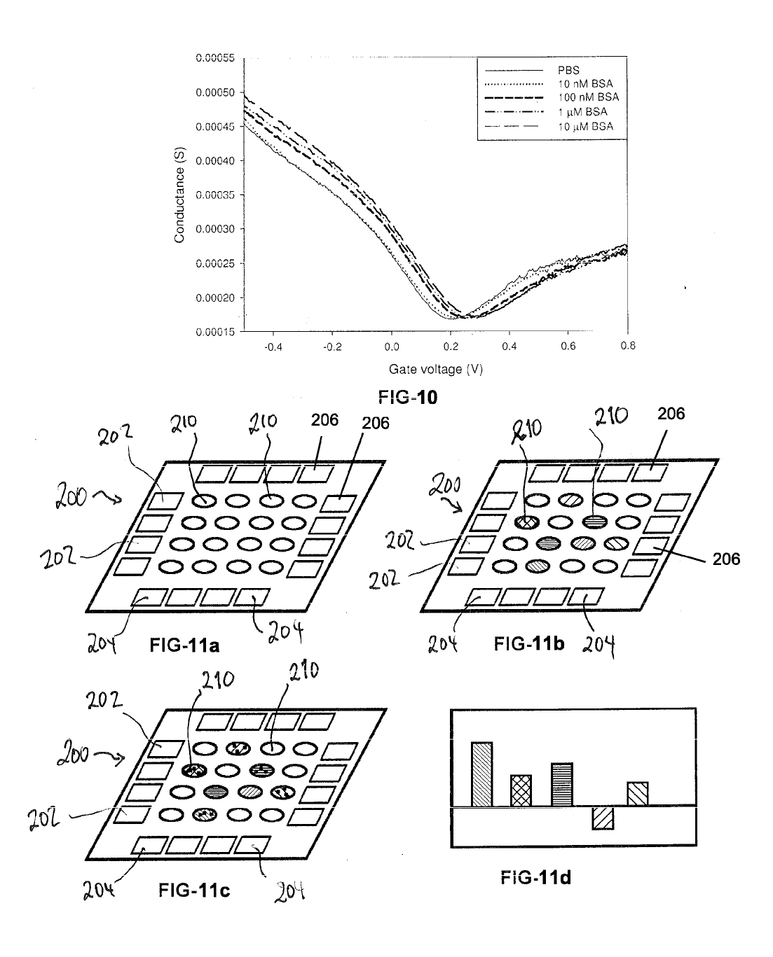

A graphene ISFET structured like the sensor 100 may also be used for label-free biomolecule detection. Several chemical reactions are available, covalent reactions and noncovalent reactions, such as p-p interaction, hydrophobic effects, and van der Waals forces. FIG. 8 illustrates how the graphene sheet 10 can be prepared to be sensitive to a specific biomolecule by covalent bonding in accordance with one embodiment of the present invention. Starting with the plain graphene sheet 10, nitric acid (HNO₃) is applied to form carboxyl groups (COOH) with individual carbon atoms 12 embedded in the honeycomb structure 16. A zero-length crosslinking agent, such as 1-ethyl-3-[3-dimethylaminopropyl]carbodiimide hydrochloride (EDC), is then used to make the graphene sheet 10 receptive to attaching affinity probes R—NH₂, where the letter R represents custom capture molecules forming the affinity probe. Affinity probes are typically monoclonal antibodies that bind only the type of biomolecule to be detected.

According to one embodiment of the invention, the immobilization of affinity probes R on the graphene sheet can be facilitated by incorporating edge defects in the graphene sheet as shown in FIGS. 14-16. The edges may make a direct attachment of antibodies possible where previously crosslinkers were required for immobilization.

After the graphene sheet 10 has been prepared for affinity to a specific biomolecule 138, FIG. 9 illustrates the process of attaching individual specific biomolecule 138 to the affinity probes R immobilized on the graphene sheet 10 according to one embodiment of the present invention. The graphene sheet 10 is exposed to a solution containing a concentration of the specific biomolecule 138. As the biomolecules 138 pass along the graphene sheet 10, the biomolecules 138 dock onto free affinity probes R and become attached to the graphene sheet 10. This attachment of the biomolecules 138 leads to a change in conductance of the graphene sheet 10, measurable between the electrodes 102 and 104. The higher the concentration of specific biomolecules 138 is in the solution, the faster will a saturation set in once most or all of the available affinity probes are occupied by a biomolecule 138. Therefore, the time at which the saturation sets in can be evaluated to determine the concentration of the specific biomolecules 138 in the solution. In a similar manner, because the change in conductance occurs at a speed that positively correlates with the biomolecule concentration, the slope of the conductance, i.e. the rate of change, is another indicator for the biomolecule concentration.

In analogy to FIG. 6, FIG. 8 shows the conductance of the graphene sheet 10 over the gate voltage at different concentrations of bovine serum albumin (BSA, from zero to about 10 μM, preferably 10 μM). BSA is mixed with physiological solution, phosphate buffer solution in a concentration of about 10 mM with about pH 7.2, preferably pH 7.2, also called 1× solution for having salt concentrations similar to biological fluids. In this experiment, the BSA is negatively charged, where the isoelectric point of the BSA is about 5.3, preferably 5.3. The Dirac point increases from about 0.2V for the pure phosphate buffer solution (PBS) to about 0.28 V for the PBS containing about 10 μM BSA, preferably 10 μM BSA. This can be explained by the fact that BSA is negatively charged.

Under different settings, for example when the gate voltage is about 0.3 V, preferably 0.3 V, which is slightly above the Dirac point, the carriers in the graphene are electrons. The conductivity decreases with increased BSA concentration, where BSA is an electron acceptor. When the applied gate voltage is about −0.4 V, preferably −0.4 V, the graphene sheet 10 becomes metallic (high carrier concentration) and the carriers are holes. Although the conductance increases with increased BSA concentration, the sensor becomes less responsive. The graphene biosensor is more sensitive to the surface charges if working around the Dirac point, where the carrier concentration is at a minimum. Accordingly, the gate voltage can be calibrated to conduct the measurements near the Dirac point. Because the Dirac point moves to higher gate voltages as the concentration of BSA increases, the Dirac point allows a quantitative determination of the BSA concentration in the physiological solution.

The targeted, label-free detection of specific biomolecules opens a multitude of possibilities for the production of graphene-based protein microarrays and their use in the simultaneous detection of multiple biomolecule concentrations.

FIGS. 9a through 9d give an illustrative schematic example of such a graphene-based microarray sensor 200. Illustrated in FIG. 11a is a microarray sensor 200 having a plurality of graphene sheets 210, each of which is connected to one of a plurality of source electrodes 202 and to one of a plurality of drain electrodes 204. Initially, all graphene sheets 210 are identical. Opposite the source electrodes 202 and the drain electrodes 2-4 are reference electrodes 206 for supplying a gate voltage. Then, as illustrated in FIG. 9b, individual graphene sheets are prepared to carry immobilized affinity probes, where different graphene sheets carry different affinity probes as indicated by different hatchings of the graphene sheets 210. A process of immobilizing affinity probes was described in connection with FIG. 8, but other processes are also available. Notably, less than all graphene sheets 210 may carry affinity probes, or a subset of the graphene sheets may carry identical affinity probes without leaving the scope of the present invention.

The immobilized affinity probes attaching to different biomolecules allow a simultaneous quantitative measurement of different biomarkers, for example biomarkers associated with different types of cancer, in blood serum or tissue extraction. The molecular events in the protein microarray 200 are complex and form multi-step processes. Now referring to FIG. 11c, the biological fluid, dissolved in a buffer solution, is applied on the graphene sheets 210 of the microarray 200. The antigens contained in the fluid diffuse in the buffer solution until the antigens find a matching antibody among the affinity probes immobilized on the individual graphene sheets. The capture of the matching antibodies by the respective immobilized antigens is rapid, so the supply of the biological fluid containing the antigens is preferably faster at lower antigen concentrations in the biological fluid in order to prevent a depletion of antigens in the fluid.

The individual graphene sheets 210 function as field-effect transistor. When charged molecules (such as antigens) are adsorbed on one of the graphene sheets 210, the carrier concentration of the respective graphene sheet 210 is reduced or increased by the charges. FIG. 11d schematically illustrates individual changes in conductance depending on a change in charge carriers. Because each of the plurality of graphene sheets 210 carries affinity probes of one specific biomolecule, the different bars depicted in FIG. 11d pertain to different identifiable biomolecules. The presence of each of these types of biomolecules can be quantified based on conductance behavior of individual electrode pairs 202 and 204. No labeling step is required because the individual affinity probes are already located in predetermined positions.

FIG. 11d illustrates a saturation curve indicative of biomolecules 238 attaching to affinity probes on one the graphene sheets 210.

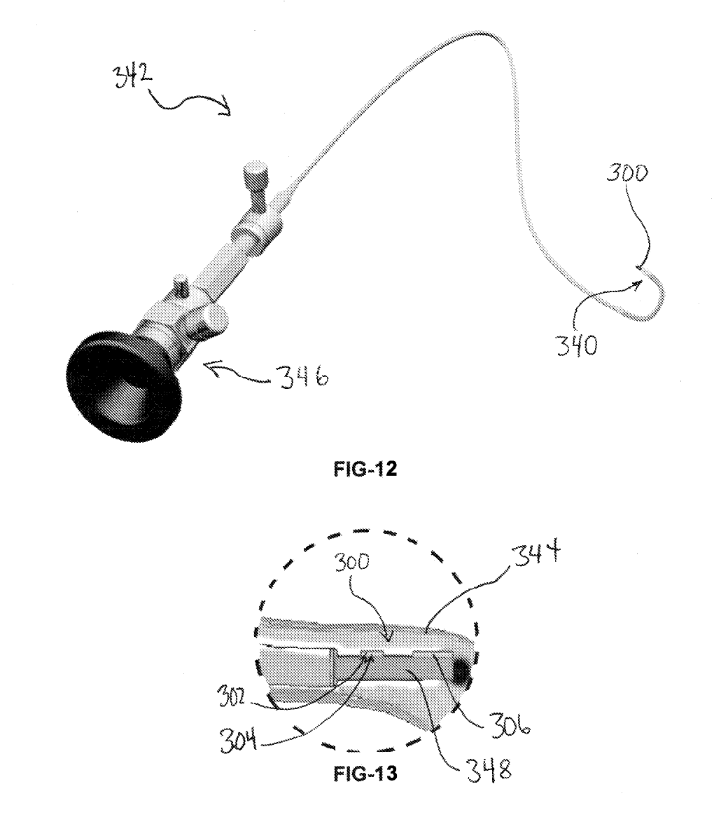

Referring now to FIGS. 12 and 13, a biosensor 300 may be mounted on a catheter 342, where the sensor 300, configured as an ISFET, is attached to a tip 340 at a distal end of the catheter 342 for determining the presence of certain biomarkers inside a body vessel 344. In the shown embodiment of the invention, the biosensor 300 is a single graphene sensor with an exposed graphene sheet as illustrated in FIG. 2. The enlarged illustration of the catheter tip 340 in FIG. 13 shows the graphene sensor 300 with a reference electrode 306 arranged proximate a source electrode 302 and a drain electrode 304 on a sensor carrier 344 attached to the tip 340 of the catheter 342. The reference electrode 306 supplies the reference gate voltage for the biosensor 300. Electric conduits (not shown) extend through the length of the catheter 342 and establish an electrical contact between the biosensor 300 on the tip 340 of the catheter 342 and a connector (not shown) on the proximal end 346 of the catheter 342. The connector is configured to connect the biosensor 300 to a power supply (not shown) and to measuring equipment (not shown). The catheter 342 allows an in situ measurement, which is especially beneficial when volatile substances must be detected that have a limited lifespan once exposed to the atmosphere. It also allows instantaneous measurement of changes occurring inside the body vessel 344.

In summary, graphene addresses the current bottleneck of label-free electrical detection by improving manufacturing cost, surface functionalization and response time. The advantages of graphene in biosensor design are evident.

Graphene is highly compatible with microfabrication techniques for device integration because of its planar structure. The surface area and geometry of graphene can be controlled using contact aligner and oxygen plasma.

Graphene is very efficient to detect antigens in extremely low concentration. Graphene is two-dimensional structure, and thus its entire surface is exposed to the solution. Using finite element analysis, it is estimated that the time for molecular binding is seconds for micron-sized graphene compared to hours for nanowires, assuming the concentration of about 10 fM, preferably 10 fM, a diffusivity of target proteins of about 10 μm²/s, preferably 10 μm²/s, a flow rate of about 10 μL/min, preferably 10 μL/min, reaction kinetics of about 10⁶ M⁻¹s⁻¹, preferably 10⁶ M⁻¹s⁻¹, and a binding site density of about 2×10¹² sites/cm², preferably 2×10¹² sites/cm². The rapid detection is significant for analyzing clinical samples, such as human blood or other body fluids, because the presence of proteolytic enzymes in the blood causes denature of proteins in a long run.

Graphene is easier to be functionalized than carbon nanowires. Because of its planar structure, various antibody molecular probes can be immobilized uniformly on the chemically-functionalized graphene using low-cost robotic spotting techniques.

Low-cost, wafer-scale, high-quality graphene has been grown using chemical vapor deposition. Compared to lithography-based silicon nanowires, the manufacturing cost of a graphene sensor is substantially lower because it does not require expensive nanolithography. Further, graphene can be transferred and integrated with polymer substrates for the applications such as flexible electronics and implantable microdevices.

While various embodiments for carrying out the invention have been described in detail, those familiar with the art to which this invention relates will recognize various alternative designs and embodiments for practicing the invention as defined by the following claims.

1. A graphene biosensor comprising:
an electrically insulating substrate;
a first metallic electrode and a second metallic electrode, the first and second metallic electrodes being mounted on the substrate;
a single-layer graphene sheet in electrical contact with and connecting the first and second metallic electrodes, the graphene sheet comprising perforations or gaps with edges having a total edge length.

2. The graphene biosensor of claim 1, wherein the perforations are holes.

3. The graphene biosensor of claim 2, wherein the holes have a diameter smaller than about 10 μm.

4. The graphene biosensor of claim 3, wherein the holes have a diameter smaller than about 5 μm.

5. The biosensor of claim 1, wherein the perforations are arranged in a substantially regular pattern at a distance from each other smaller than about 5 μm.

6. The biosensor of claim 1, wherein the perforations are arranged in a substantially regular pattern at a distance from each other smaller than about 1 μm.

7. The biosensor of claim 1, wherein the graphene sheet has an area and the total edge length relative to the graphene sheet area has an edge-to-area ratio above 0.1 μm⁻¹.

8. The biosensor of claim 7, wherein the edge-to-area ratio is greater than about 0.5 μm⁻¹.

9. The biosensor of claim 7, wherein the edge-to-area ratio is greater than about 0.7 μm⁻¹.

10. The biosensor of claim 1, further comprising a reference electrode configured to be supplied with a variable gate voltage and configured to be in indirect contact with the graphene sheet via a fluid connection.

11. The biosensor of claim 1, wherein the graphene sheet comprises immobilized affinity probes attached to the edges of the perforations and configured to attach specific molecules to the graphene sheet.

12. The biosensor of claim 11, wherein the affinity probes are antibodies configured to attach specific antigens to the graphene sheet.

13. The biosensor of claim 1, wherein the graphene sheet is part of an array of at least two graphene sheets including a first and a second graphene sheet.

14. The biosensor of claim 13, wherein both the first graphene sheet and the second graphene sheet comprise immobilized affinity probes.

15. The biosensor of claim 14, wherein the affinity probes associated with the first graphene sheet are different than the affinity probes associated with the second graphene sheet.

16. The biosensor of claim 13, wherein at least one of the at least two graphene sheets comprises immobilized affinity probes and at least one of the at least two graphene sheets is free of any affinity probes.

17. The biosensor of claim 1, wherein the biosensor is configured as an ion-sensitive field effect transistor.

18. The biosensor of claim 17, wherein the biosensor is configured to measure a property of a liquid contacting the reference electrode, the source electrode, the drain electrode and the first graphene sheet, the biosensor measuring a current between the drain electrode and the source electrode during exposure to the liquid.

19. The biosensor of claim 18, wherein the biosensor is calibrated to operate near the Dirac point of the conductance during exposure to the liquid.

20. The biosensor of claim 18, further comprising a cavity and at least two ports in fluid communication with the cavity, the cavity containing the graphene sheet and the set of electrodes, and the ports being configured to supply the liquid to the cavity and to drain the liquid from the cavity.

21. The biosensor of claim 20, wherein one of the at least two ports is an inlet port for supplying the liquid to the cavity and another one of the at least two ports is an outlet port for draining the liquid from the cavity, both inlet port and outlet port being configured to be operated at the same time and to allow a continuous flow of liquid through the cavity.

22. The biosensor of claim 1, wherein the biosensor is configured to be mounted on a catheter of the type having a proximal end and a distal end, an electric connector disposed at the proximal end, and an electrical connection extending along the catheter and connecting the distal end to the electric connector, the biosensor being configured to be mounted on the distal end and to be connected to the electrical connection.
